(19) United States
(12) Patent Application Publication
(10) Pub. No.: US 2026/0125626 A1
(43) Pub. Date: May 7, 2026

(54) STRUCTURED REACTOR FOR BIOFILM CULTIVATION AND GASEOUS EXTRACTION

(71) Applicant: H2Biosys Inc., Dover, DE (US)

(72) Inventors: Nicole Billings, Dover, DE (US); Piro Siuti, Framingham, MA (US); Mohamad Sater, Dover, DE (US)

(21) Appl. No.: 19/377,946

(22) Filed: Nov. 3, 2025

Related U.S. Application Data
(60) Provisional application No. 63/715,956, filed on Nov. 4, 2024.

(51) Int. Cl. C12M 1/107; C12M 1/00; C12M 1/12; C12M 3/00

(52) U.S. Cl. CPC C12M 21/04; C12M 23/42; C12M 23/48; C12M 25/02; C12M 29/06; C12M 47/18

(57) ABSTRACT

A scalable and modular bioreactor defines a compact gas-liquid interface area populated with gas consuming microbes in a stacked arrangement for facilitating growth of the microbes in the presence of the greenhouse gases the microbes consume. A biofilm-based interface between liquid and gaseous regions of the reactor positions the microbes at the gas-liquid interface for metabolic exchanges of the gas for conversion into liquid and gaseous output. Microbes are selected for an affinity for a particular gas, such as methane (CH₄), and metabolize carbon based outputs such as carbon biopolymers and gases such as carbon dioxide (CO₂) and hydrogen (H₂) for harvesting or recircling back for further metabolic conversions.

### RELATED APPLICATIONS

This patent application claims the benefit under 35 U.S.C. § 119 (e) of U.S. Provisional Patent App. No. 63/715,956, filed Nov. 4, 2024, entitled “STRUCTURED REACTOR FOR BIOFILM CULTIVATION AND GASEOUS EXTRACTION,” incorporated herein by reference in entirety.

### BACKGROUND

Substantial attention has been focused on harmful effects of so-called “greenhouse” gases (GHG). Anthropogenic gases emitted by various sources contribute to an unhealthy atmospheric aggregation and exacerbation of climate change. Human activities have intensified the greenhouse effect, leading to elevated global temperatures, driving climate change. Methane (CH₄), a GHG 80x more potent than CO₂, continues to rise at an alarming rate. The U.S. alone releases 800 million metric tons CO₂ equivalent (MMT CO2-e) from sources ranging from municipal landfills, agricultural waste, and fossil fuel extraction.

### SUMMARY

A scalable and modular bioreactor defines a compact gas-liquid interface area populated with gas consuming microbes in a stacked arrangement for facilitating growth of the microbes in the presence of the greenhouse gases the microbes consume. A biofilm-based interface between liquid and gaseous regions of the reactor positions the microbes at the gas-liquid interface for metabolic exchanges of the gas for conversion into liquid and gaseous output. Microbes are selected for an affinity for a particular gas, such as methane (CH₄), and metabolize carbon based outputs such as carbon biopolymers and gases such as carbon dioxide (CO₂) and hydrogen (H₂) for harvesting or recircling back for further metabolic conversions.

Configurations herein are based, in part, on the observation that greenhouse gas mitigation, and in particular methane mitigation, can be offset by conversion from carefully selected microbes that consume and metabolize the respective greenhouse gases. Unfortunately, conventional approaches to greenhouse gas mitigation suffer from the shortcoming that conventional approaches cannot scale appropriately because an area of the gas-liquid interface though which the microbes thrive is insufficiently small. Batch and liquid reactors define a gas-liquid interface only on the surface of a large liquid volume, leaving metabolizing microbes mostly submerged and distant from the gas-liquid interface, often under pressure to encourage gaseous dissolution in the liquid volume. Accordingly, configurations herein substantially overcome the shortcomings of conventional greenhouse gas mitigation by providing a scalable reactor approach that expands an area of the gas-liquid interface beyond the reactor footprint for ensuring that the microbial population is maintained at the gas-liquid interface such that the microbes are engaged with a gaseous side of the reactor for metabolism of the target gas, such as methane.

The disclosed approach employs a scalable and modular bioreactor such that a compact gas-liquid interface area is populated with gas consuming microbes in a stacked arrangement for facilitating growth of the microbes in the presence of the greenhouse gases the microbes consume. In further detail, a particular configuration deploys a bioreactor containment device having a containment unit defining a liquid-gas interface, and a growth substrate disposed at the liquid-gas interface and having a liquid exposed side and a gas exposed side. The growth substrate provides cultivation of a microbial product from gaseous engagement with the gas-exposed side of the growth substrate at the liquid-gas interface for efficient aerobic metabolism based on amble exposure to the target gas.

### BRIEF DESCRIPTION OF THE DRAWINGS

The foregoing and other features will be apparent from the following description of particular embodiments disclosed herein, as illustrated in the accompanying drawings in which like reference characters refer to the same parts throughout the different views. The drawings are not necessarily to scale, emphasis instead being placed upon illustrating the principles of the invention.

FIG. 1 is a context view of a general bioreactor configuration in a gas mitigation environment suitable for use with configurations herein;

FIGS. 2A-2C are schematic diagrams of a bioreactor in the environment of FIG. 1;

FIGS. 3-3A are a side elevation of an example bioreactor structure according to FIGS. 1-2C;

FIG. 4 is a schematic diagram of cassette removal in the reactors of FIGS. 2A-2C;

FIG. 5 shows modularity and scaling of the bioreactors of FIGS. 1-4;

FIG. 6 shows an apparatus for gathering the biomass generated in the reactors of FIGS. 1-5;

FIG. 7 shows an example of biofilm generation in the bioreactor of FIGS. 1-6 using methane; and;

FIG. 8 shows an example of biofilm generation in the bioreactor of FIGS. 1-6 using carbon dioxide.

### DETAILED DESCRIPTION

Configurations herein depict an example of a bioreactor including a modular and scalable arrangement of a plurality of growth substrates oriented at a liquid-gas interface for facilitating growth of a microbial, biofilm product generated from conversion of harmful or undesirable gaseous discharge such as methane (CH₄), carbon dioxide (CO₂) and carbon monoxide (CO).

Throughout the natural world, methane sinks are a key part of carbon cycling. Notably, a diverse group of prokaryotes play a significant role in this process due to their direct consumption of methane as a carbon source. Methanotrophic bacteria (MOB) occupy many different mesophilic and extreme ecosystems (e.g., wetlands, freshwater and marine sediments, tundra, acidic hot springs, soil). MOBs are also naturally found in air/fluid interface biofilm communities around CH₄ rich aquatic environments, allowing for direct absorption of CH₄ from biogenic processes under aerobic or microaerophilic conditions. Importantly, MOBs metabolize CH₄ into valuable byproducts, such as hydrogen (H₂) gas and carbon-based biopolymers. While the ecological importance of these microbes is well-recognized, their industrial potential remains generally underexplored due to inefficient mass transfer of insoluble CH₄ and scalability limitations of current bioreactor technologies. Furthermore, other MOB technologies have focused on broad CH₄ sequestration direct from the atmosphere at extremely low concentrations (<500 ppm CH₄), which are economically impractical due to the number, scale, and high operational cost of reactors deployed away from CH₄ point sources. A unique and robust approach as defined herein to capture methane directly at anthropomorphic point sources provides a beneficial impact on climate health.

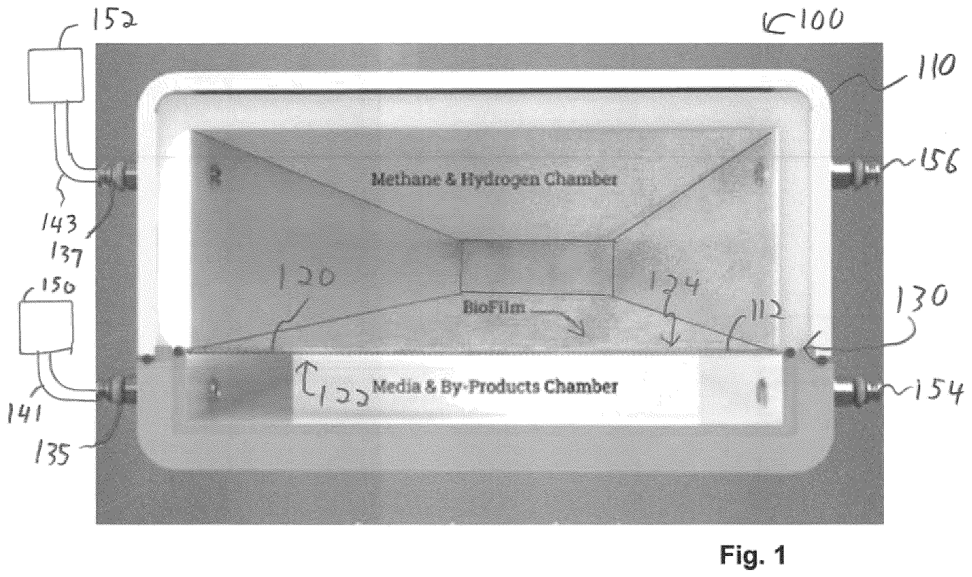

FIG. 1 is a context view of a general bioreactor configuration in a gas mitigation environment suitable for use with configurations herein. Referring to FIG. 1, a bioreactor containment 100 includes a containment unit 110 defining a liquid-gas interface 112, and a growth substrate 120 disposed at the liquid-gas interface 112 which has a liquid exposed side 122 and a gas exposed side 124. The growth substrate 120, typically a porous membrane, provides cultivation of the biofilm 130 as a microbial product from gaseous engagement with the gas-exposed side 124 of the growth substrate at the liquid-gas interface 112. In other words, the microbial product 130 results from gaseous methane on the gas exposed side 124, and need not rely on dissolved methane in liquid. The use of gaseous methane or other reactants allows a higher volume of methane to be handled, as it is not limited to merely the volume of methane dissolved in the water or other liquid, and also does not require the higher pressure employed by conventional approaches to increase the amount of dissolved methane. Any suitable aerobic process may be performed in the bioreactor containment 100.

In FIG. 1, the supply of gaseous and aqueous products allows a continuous mode of operation. Configurations herein may be implemented in either batch or continuous modes for generating the microbial product 130 on the growth substrate using available carbon based gases (CH₄. CO₂ and CO₂). The containment unit 110 includes a liquid coupling 135 between the liquid exposed side and a liquid source, a gaseous coupling 137 between the gas exposed side and a gaseous source, such that the gaseous engagement provides a combination of a liquid from the liquid source and a gas from the gaseous source in communication with the growth substrate 120. Complete throughput further employs a liquid vessel 141 from a liquid source 150 in fluidic communication with the liquid exposed side 122 and a gaseous vessel 143 from a gaseous source 152 in fluidic communication with the gas exposed side 124. A complementary liquid product outflow 154 and a gaseous product outflow 156 gathers beneficial yields from the containment resulting from continuous microbial growth based on the respective liquid and gaseous flow. The membrane may be of a predetermined porosity for encouraging microbial activity on the gaseous side 124 while passing moisture and nutrients from the liquid. PVDF (Polyvinylidene fluoride) or other suitable membrane material may be employed.

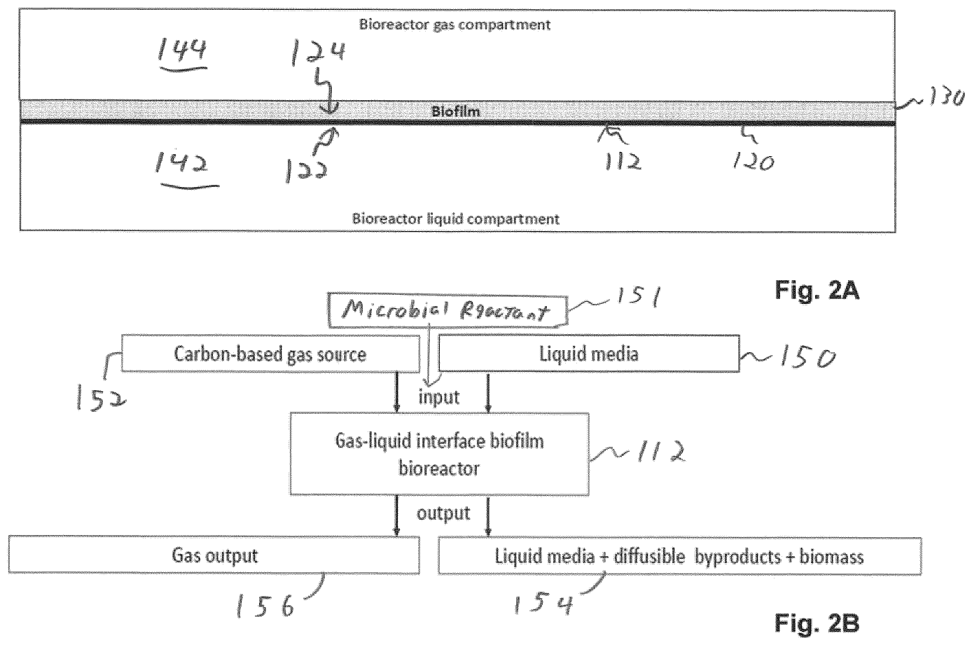

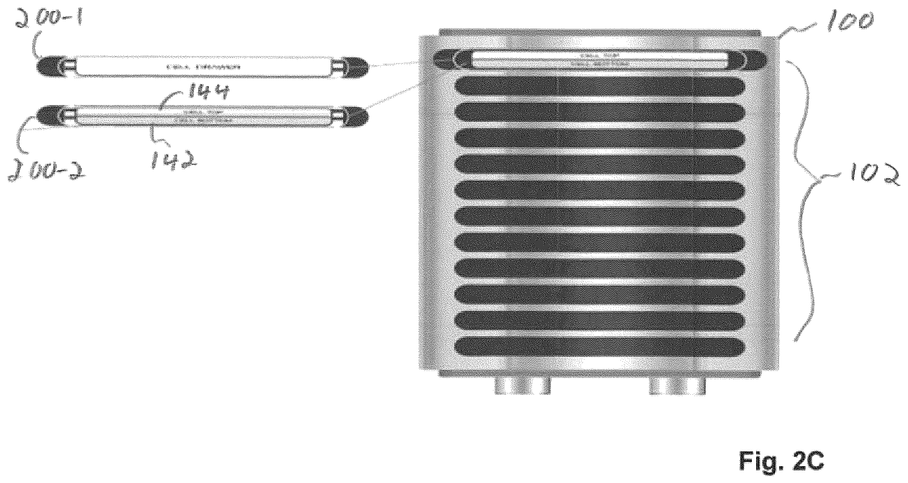

FIGS. 2A-2C are schematic diagrams of a bioreactor in the environment of FIG. 1. Referring to FIGS. 1 and 2A-2C, FIG. 2A shows a typical configuration in which the growth substrate 120 is disposed in a substantially horizontal orientation, where the gas exposed side 124 and gas 144 are disposed above the liquid exposed side 122 containing the liquid 142. The growth substrate 120 is ideally positioned at the top of the liquid 142 for accumulating a cultivated biofilm 130 from contact with both the liquid 142 and the gas 144. In other words, the growth substrate 120 is positioned at the gas-liquid interface for favoring accumulation of the biofilm 130 on the gaseous side from engagement and reaction with a gaseous phase of reactants. The accumulated, grown biofilm 130 is subsequently separable from the growth substrate 120 for harvesting the microbial product.

FIG. 2B is a block diagram of the process in FIGS. 1-2A. The gaseous source 152, liquid source 150, and a microbial reactant 151 to start or seed the biofilm 130 generation are combined in one or more containment units 110 within the bioreactor containment 100. The liquid-gas interface 112, being co-located with the growth substrate 120, provides ideal conditions for biofilm 130 generation. The microbial reactant 151 is disposed on the growth substrate 120, and in a typical configuration the gaseous source 152 is methane and the liquid source 150 is water. Alternate gases, liquids and reactants 151 may be provided.

In the bioreactor containment 100, the area of the growth substrate 120 defines the available region for the biofilm 130 growth. Accordingly, FIG. 2C demonstrates a scalability and extension of the growth substrate 120 formed in each of a series or stack of the containment units 110 defined by exchange units 200-1 . . . 200-N (200 generally) in the bioreactor containment 100. A plurality of exchange units 200 are stacked in a parallel adjacency in the containment, such that each of the exchange units 200 defines the liquid-gas interface 112 between the liquid 142 and the gas 144. A further enhancement is each exchange unit 200 is formed as a removable cassette, where each cassette is slidably insertable and removable from a respective slot 102 in the bioreactor containment 100.

The gas-liquid interface 112 in the bioreactor containment 100 is therefore designed to support multiple containment unit 110 systems defined by the cassettes. Each cassette (exchange unit 200) in the reactor is comprised of a gas 144 compartment and a liquid 142 compartment. These compartments are separated by a membrane forming the growth substrate 120. Microbial cells or reactants 151 are seeded on the gaseous side 124 in the gas compartment. A typical membrane is comprised of a porous material, such that the pore diameter in the membrane is too small for microbes to pass into the liquid chamber. The microbes grow as a biofilm 130 on the porous membrane, capturing carbon gas and acquiring additional nutrients from the liquid 142 that permeates the membrane defining the gas-liquid interface 112.

The cassette structure forming the exchange units 200 in the bioreactor containment 100 provide a novel gas-liquid interface biofilm reactor that leverages the metabolic capabilities of MOBs to convert CH₄ into H₂ gas and carbon biopolymers on an industrial scale. A particular advantage is provided by the integration of MOB natural biofilm growth modalities at gas-liquid interfaces with the disclosed reactor design to maximize feedstock uptake. This approach minimizes common problems with conventional industrial reactor designs such as accessibility to CH₄ gas, high energy consumption required for large scale batch mixing, susceptibility to contamination, and high maintenance costs that prevents deployment of conventional reactor systems to CH₄ source points. Further, the pioneering reactor design employs a method of aerobic biofilm cultivation within individual cassettes that enables a modular, scalable solution for multiple customer bases. Unlike conventional methane abatement approaches that require fixed facilities and high-cost system overhauls at methane point sources, the disclosed approach offers a flexible, mobile deployment system with standardized scalable units.

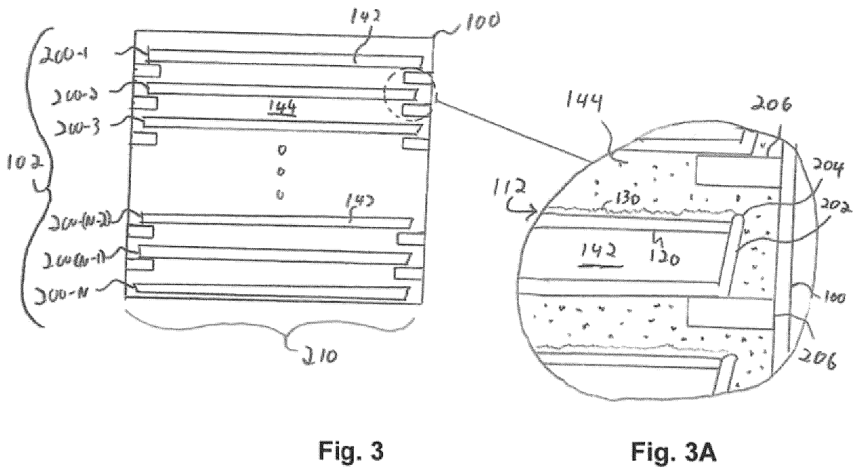

FIGS. 3-3A are a side elevation of an example bioreactor containment 100 and cassette structure according to FIGS. 1-2C. Referring to FIGS. 1-3A, an alternate configuration employs a single gas 144 volume in the containment unit 110. The containment unit 110 encapsulates a common gaseous volume 144 in fluidic communication with each of the cassettes, and each exchange unit 200 need only encapsulate or contain the liquid 142 while the gas 144 fills the common volume inside the bioreactor containment 100. The exchange units 200 take the form of an open tray 202, and the growth substrate 120 extends across the tray to meet the tray wall at or just below a lip 204, and therefore flush or almost flush with an open perimeter of the tray 202. Support planes 206 may take the form of a rigid strip along the edge, or span the entire width 210, optionally defined by one or more bars or dowels to define a “rack.” In either design, the entire interior volume defines a single fluid vessel for the gas 144 and separate fluid 142 volumes in each of the trays 202. Accumulating biofilm 130 may be easily removed by clearing or scraping the flush surface of the growth substrate 120.

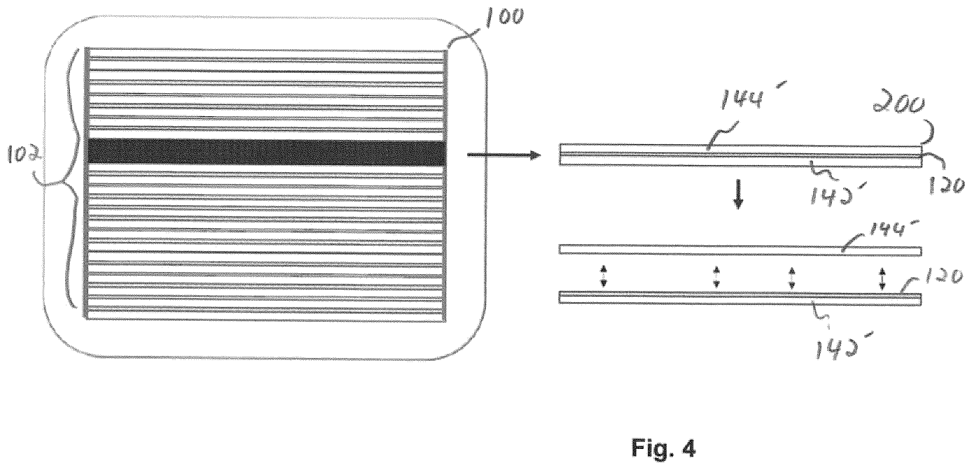

FIG. 4 is a schematic diagram of cassette removal in the reactors of FIGS. 2A-2C. Each cassette within a bioreactor unit 100 is individually removable. Once removed from the reactor, the cassette compartments can be separated to reveal the microbes in biofilm 130 form on the membrane. Biomass can be collected for further processing. Another clean cassette can replace the open position in the reactor. Each of the cassettes therefore defines a respective sealed fluid volume 124′ separated from the others of the respective sealed fluid volumes by the growth substrate 120 membrane. A sealed gas volume 144′ effectively forms a layered structure or compartment, separable from the fluid 124 and the growth substrate 120 for biofilm harvesting, or the gas volume may be the open interior of the bioreactor containment 100, as in FIG. 3.

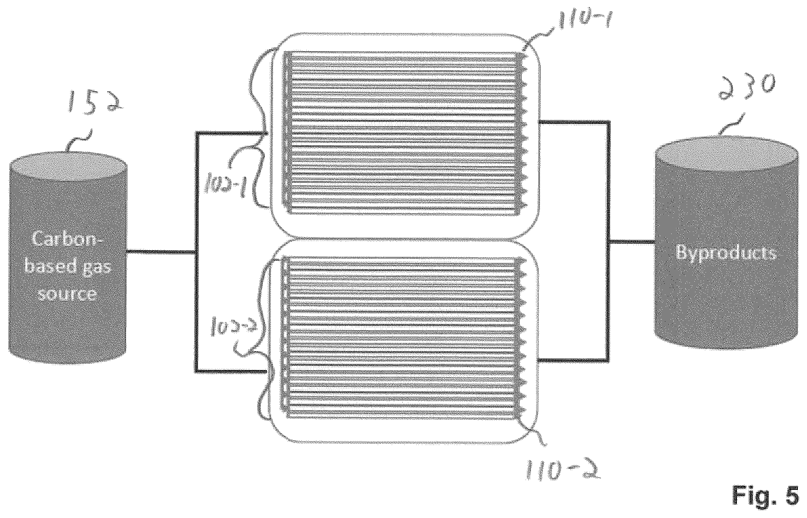

FIG. 5 shows modularity and scaling of the bioreactors of FIGS. 1-4. A paramount feature is the scaling of the effective growth substrate 120 area by vertically stacking the trays/exchange units 200 in the slots 102-1 . . . 102-2 (102 generally) of each containment 110, and by introducing multiple containment units 110-1 . . . 110-2 (110 generally). Once attaching at least the gaseous source 152 and other needed liquid sources, the byproducts 230 of liquid 142 and accumulated biofilm 130 are harvested.

Conventional packed-bed biofilm reactors commonly used in wastewater treatment or biofiltration are impractical for CH₄ capture due to limited mass transfer with biofilm submerged in growth media. CH₄ is highly insoluble in aqueous solution, resulting in the need for an alternative method to enhance mass transfer. The gas-liquid interface strategy of the disclosed approach exhibits an example configuration where MOB biofilms grow on a wetted surface enhancing accessibility to CH₄. In practice, it is expected that minimal biofouling and cell dispersal occurs throughout the reactor as MOB are cultivated in multiple cassette chambers. Other bacteria that may be employed include Bacillus subtilis and Cyanobacteria strains. Suitable strains that may be cultivated in a biofilm form and used for a process called biosorption from the liquid chamber. The walls of these bacteria have negatively charged functional groups that can bind the positively charged rare earth minerals. This is especially the case for Bacillus and Terbium. Other examples include acidophilic bacteria in a biofilm that can release acids which break down minerals that contain rare earth minerals and release the rare earth minerals for subsequent processing. A further example is Methylotrophs, a bacteria that is being researched for rare earth mineral capturing since they produce and release a specific protein that binds to rare earth minerals. Additionally, the bioreactor unit 100 is designed for efficient capture of gas, soluble carbon byproducts, or biomass as revenue generating commodities, several of which are depicted below.

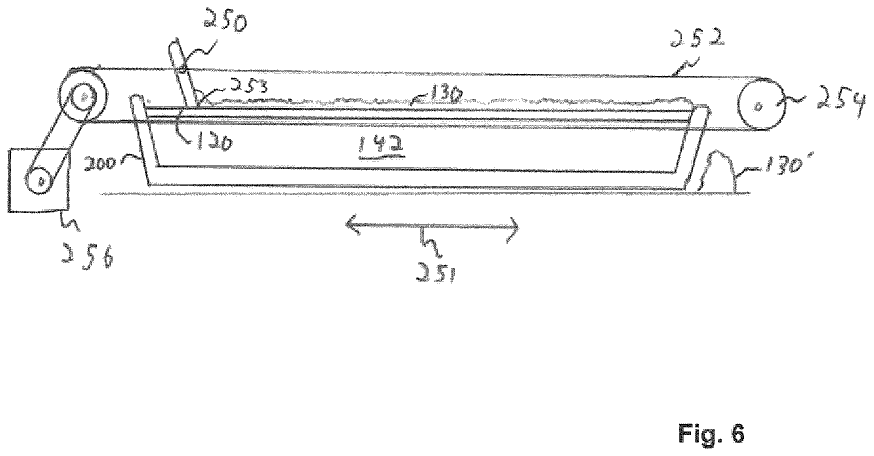

FIG. 6 shows an example apparatus for gathering the biomass 130 generated in the reactors of FIGS. 1-5. Referring to FIGS. 1-5, the accumulating biofilm 130, facilitated by gaseous activity at the air-gas interface 112, may be harvested from the growth substrate by any suitable manual or automated approach. In a particular configuration, the growth substrate 120 is responsive to an elongated scraper 250 extending along the growth substrate 120 in a first dimension, and disposable across the growth substrate 120 along a second dimension 251. Physical removal of accumulated biofilm 130 allows harvesting based on a layered thickness of the biofilm. Aerobic activity resulting from microbes exposed to both moisture from the liquid 142 in the presence of the gas 144 encourages the biofilm 130 growth and accumulation. As the biofilm 130 later thickens, emerging grown becomes more distant from the liquid 142, eventually diminishing growth.

The example of FIG. 6 employs an actuator such as a belt drive 252 engaged with the scraper 250, in which the actuator is configured for advancing the scraper 250 in slideable communication with the growth substrate 120, thereby accumulating the microbial product along a leading edge 253 of the scraper 250. Any suitable motive capability may be employed, such as an electric motor 256 driving pulleys 254 around which the belt is engaged. Linear and/or magnetic actuators, for example, may also be employed. Upon attaining a distal limit of the exchange unit 200, a biofilm yield 130′ simply drops for harvesting. Scraper 250 iterations may be repeated at regular intervals based on a thickness of the biofilm 130 accumulated over time.

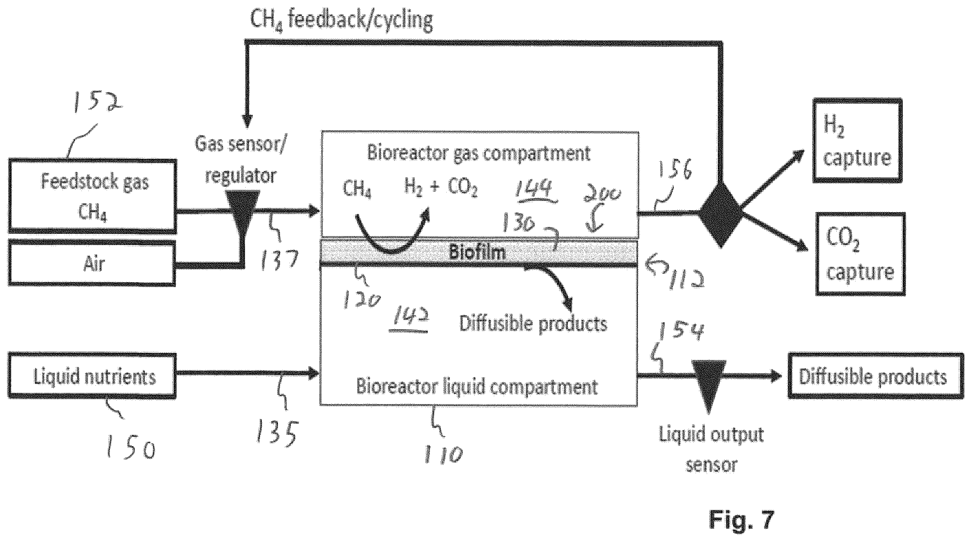

FIG. 7 shows an example of biofilm generation in the bioreactor of FIGS. 1-6 using methane. Referring to FIGS. 1-7, a particularly beneficial target gas 144 of the disclosed approach is methane. Any suitable gas, however, may be employed based on the seeded microbes and expected anerobic reaction with the gas 144 and the liquid 142 at the liquid-gas interface 112. FIG. 7 depicts the biofilm reactor single cassette operation with CH₄ feedstock converted into valuable metabolic products captured as outputs from the liquid 142 and gas 144 compartments. The gas supply 152 provides waste methane while liquid nutrients provide the liquid source 150, typically water and other nutrients based on the microbes seeded on the growth substrate 120. As the methane reacts with the biofilm 130 to produce H₂ and CO₂, diffusible products from the biofilm synthesis/reactions with methane, mostly carbon based chemical byproducts, populate the liquid 142 for subsequent harvest through the product outflow 154.

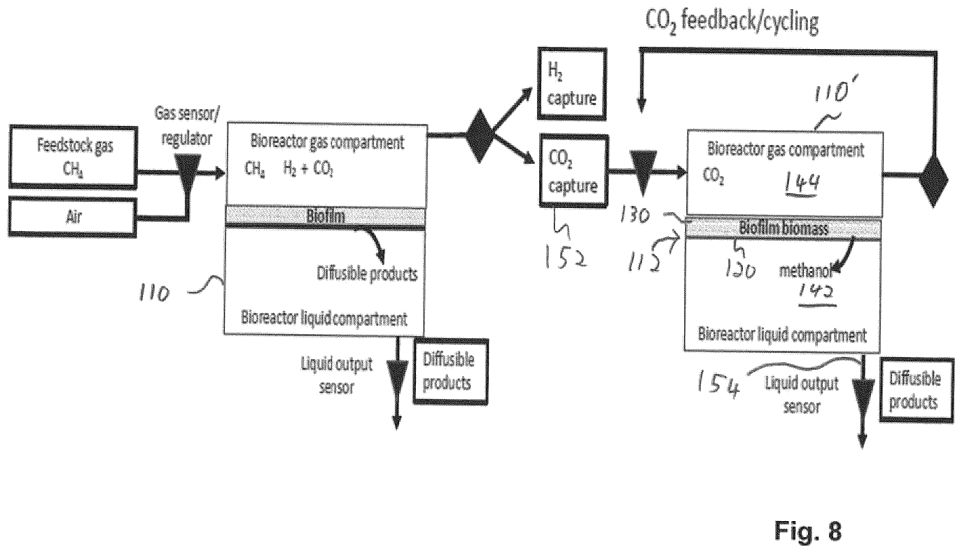

FIG. 8 shows an example of biofilm generation in the bioreactor of FIGS. 1-6 using carbon dioxide. FIG. 8 depicts an expanded system which accepts carbon dioxide CO₂ feedstock in a downstream unit 110′ for conversion into valuable biomass as a raw carbon material and methanol.

TABLE I

Methanol
Ectoine
Biopolymers (Polyhydroxy butyrate—PHB)
Succinate
Cadaverine
Hydrogen
Polysaccharides
Animal feed (Single Cell Protein—SCP)
Biodiesel precursors
Fatty acids
Lipids
Putrescine
Different acids
Crotonic acid
Butyric acid
Hydroxypropionic acid
Butanediol

Additional compounds that may be derived from the biofilm based approach herein include compounds such as those listed in Table I.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A bioreactor containment device, comprising:
a containment unit defining a liquid-gas interface;
a growth substrate disposed at the liquid-gas interface and having a liquid exposed side and a gas exposed side; and
the growth substrate providing cultivation of a microbial product from gaseous engagement with the gas-exposed side of the growth substrate at the liquid-gas interface.

2. The device of claim 1 further comprising a plurality of exchange units in the containment, the plurality of exchange units stacked in a parallel adjacency in the containment, each of the exchange units defining a respective liquid-gas interface.

3. The device of claim 1 wherein the growth substrate is disposed in a substantially horizontal orientation, the gas exposed side above the liquid exposed side, the growth substrate accumulating a cultivated biofilm, the biofilm separable from the growth substrate for forming the microbial product.

4. The device of claim 1 wherein the growth substrate is responsive to an elongated scraper, the elongated scraper extending along the growth substrate in a first dimension and disposable across the growth substrate along a second dimension.

5. The device of claim 1 further comprising an accumulation of a biofilm, the biofilm accumulating on the gaseous side from engagement and reaction with a gaseous phase of reactants.

6. The device of claim 2 wherein each exchange unit is defined by a cassette, each cassette insertably removable from the containment.

7. The device of claim 1 further comprising
a liquid coupling between the liquid exposed side and a liquid source;
a gaseous coupling between the gas exposed side and a gaseous source;
the gaseous engagement providing a combination of a liquid from the liquid source and a gas from the gaseous source on the growth substrate.

8. The device of claim 2 further comprising:
a liquid vessel in fluidic communication with the liquid exposed side;
a gaseous vessel in fluidic communication with the gas exposed side;
a liquid source engaged with the liquid vessel; and
a gaseous source engaged with the gaseous vessel, the containment operable for continuous microbial growth based on liquid and gaseous flow.

9. The device of claim 6 wherein each of the cassettes defines a respective sealed fluid volume, the sealed fluid volumes separated from the others of the respective sealed fluid volumes, the containment encapsulating a common gaseous volume in fluidic communication with each of the cassettes.

10. The device of claim 8, further comprising a microbial reactant disposed on the growth substrate, wherein the gaseous source further comprises methane and the liquid source further comprises water.

11. The device of claim 4, further comprising an actuator engaged with the scraper, the actuator configured for advancing the scraper in slidable communication with The growth substrate, thereby accumulating the microbial product along a leading edge of the scraper.

12. The device of claim 1 wherein the growth substrate is a membrane seeded with methanotrophic bacteria (MOB) for conversion of methane, the methane forming gaseous products including hydrogen and carbon dioxide, and the liquid side receives carbon-based biopolymers, the liquid side engaged with a downstream consumer of carbon based products.

13. A method for mitigation of waste gases, comprising:
deploying a containment unit defining a liquid-gas interface in an adjacency with a gaseous source for mitigation;
seeding a growth substrate with a microbial growth, the growth substrate disposed at the liquid-gas interface and having a liquid exposed side and a gas exposed side;
introducing a liquid into the containment unit in communication with the growth substrate at the gas-liquid interface; and
introducing a gas into the containment in communication with the growth substrate, the growth substrate providing cultivation of a microbial product from gaseous engagement with the gas-exposed side of the growth substrate at the liquid-gas interface.

14. The method of claim 13, further comprising deploying a plurality of exchange units in the containment unit, the plurality of exchange units stacked in a parallel adjacency in the containment, each of the exchange units defining the liquid-gas interface.

15. The method of claim 14 wherein each of the exchange units is defined by a cassette in removable engagement with the containment unit.

16. The method of claim 15 wherein the containment unit defines a common gaseous fluid volume in communication with each of the cassettes in the containment unit, each of the cassettes containing a respective liquid volume.
